(19) United States
(12) Patent Application Publication
(10) Pub. No.: US 2013/0131247 A1
(43) Pub. Date: May 23, 2013

(54) LUBRICANT COMBINATION FOR THERMOPLASTICS PROCESSING

(71) Applicants: Peter Daute, Beverstedt (DE); Thomas Schweichler, Loxstedt (DE)

(72) Inventors: Peter Daute, Beverstedt (DE); Thomas Schweichler, Loxstedt (DE)

(73) Assignee: EMERY OLEOCHEMICALS GMBH, Düsseldorf (DE)

(21) Appl. No.: 13/813,858

(22) PCT Filed: Aug. 1, 2011
(86) PCT No.: PCT/EP2011/003846

§ 371 Date: Feb. 1, 2013

(30) Foreign Application Priority Data
Aug. 2, 2010 (DE) 10 2010 033 035.3

(51) Int. Cl. C08K 5/10; C07C 67/02

(52) U.S. Cl. CPC C08K 5/10; C07C 67/02
USPC 524/313; 560/204

(57) ABSTRACT

The present invention relates to a thermoplastic composition comprising a) at least one thermoplastic polymer, b) at least one lubricant, and c) optionally further additives, the lubricant being a reaction mixture which is obtainable by the reaction of a component comprising saturated triglycerides with a primary alcohol. The present invention also relates to a process for preparing a thermoplastic composition, to the thermoplastic composition obtainable by this process, to a process for producing a moulding based on a thermoplastic composition, to a moulding obtainable by this process, and to the use of a reaction mixture.

The present invention concerns a thermoplastic compound, a procedure to manufacture a thermoplastic compound, the thermoplastic compound obtained by this process, a procedure to manufacture a mould Based on a thermoplastic compound, a mould obtained through this process as well as the application of a reaction mixture.

Thermoplastic polymers are very popular when moulds of all types are manufactured. Normally the thermoplastic polymers are processed in several steps, whereby at least one step is heating the polymers; however the heating process is frequently brought to a temperature where the thermoplastic polymer features a rheological profile sufficient for further processing. Further processing steps often incorporate intensive blending of thermoplastic polymers and other content materials and also shear strain on the polymer. The further processing steps, for example, use extrusion to achieve intensive blending of the different materials used to make a mould from thermoplastic polymer and other content materials.

To improve the mould-making characteristics, such mixtures of thermoplastic polymers and other content materials are frequently enhanced by adding a so-called lubricant. Such a lubricant is usually a low molecular compound from a group of fatty acids, fatty acid esters, wax ester, fatty alcohol ester, amide waxes, metal carboxylate, montanic acids, montanic acid ester or such high molecular compounds, as paraffines or polyethylene waxes. Polyvinylchloride especially is only processed thermoplastically with the addition of lubricant. This is particularly relevant for thermoplastic processing of hard PVC, meaning PVC masses which have no or very low plasticizer content. When using lubricants, a distinction is made between so-called “external lubricants” and “internal lubricants”. An overview on the application of lubricants is given by Worschech, K in: Becker, G.; Braun, D.; (editor) Kunststoff Handbuch (handbook of synthetic materials), 2^{nd} ed., vol. 2/1:Polyvinylchloride (1986), page 570 ff.

Lubricants which are effective as separators between the plastic mass and the processing tool metal are called “external lubricants”. The lubricant is to prevent the melted synthetic material sticking to the hot metal tools during processing, for example to the conveyor screws of the extruders or metal rollers of supercalenders. Examples for externally effective lubricants (also called “mould separating agents”) are montanic wax esters, as described in the DE-A 36 43 968 or the EP-A 346717, fatty acid complex esters of polyol, dicarbon acids and fatty acids, metal saponificants such as heavy metal stearate, paraffin wax or paraffin oil, polyethylene wax or amide wax such as ethylene diamine distearate.

“Internal lubricants” are lubricants which facilitate liquidity of the melted material. Examples for internally effective lubricants are esters of stearin acids such as butyl stearate, fatty acid partial ester of glycerine such as glycerine monooleate or phthalale acid esters of fatty alcohols such as distearyl phthalate.

For reasons of economy and ecology the direct use of saturated triglycerides such as hardened tallow or hardened rapeseed oil as lubricant would be preferable. These compounds however are effective externally (refer Becker/Braun, Kunststoff Handbuch (handbook of synthetic materials, vol. 2/1, Carl Hanser Verlag 1986 page 570-595) and cannot used as such. The application of admixtures containing saturated triglycerides with other synthetic additives is described in WO-A-2005/082991.

The present invention is based on the task to overcome at least in part the disadvantages of the present status with respect to a thermoplastic mould mass.

Especially the invention was based on the task, to identify a lubricant for thermoplastic compounds, which is based on sustainable raw materials and therefore advantageous from an ecological point of view and at the same time features a very pronounced internal lubricant effect. Moreover, this lubricant is to be manufactured easily from sustainable raw materials.

Also, the lubricant was supposed to be very compatible especially with polyvinylchloride.

Furthermore, the lubricant or a thermoplastic compound containing the lubricant was supposed to feature a very high thermal stability.

A contribution to solving the task mentioned earlier is made by a thermoplastic compound containing
a) A minimum of one thermoplastic polymer
b) A minimum of one lubricant
c) Possibly further content materials
   Whereby at least one lubricant is a reaction mixture, which has been preferably obtained by reacting a component containing saturated triglyceride with a primary alcohol.

Surprisingly it was found that saturated triglycerides, un-estered with primary alcohols, may be used as internal lubricants in processing thermoplastic synthetic materials, without negatively impacting the rheological characteristics. Additionally, these products improve the achievable thermal stability of the finished products. The lubricants consist of a high percentage of sustainable raw materials and are therefore ecologically advantageous. Moreover, manufacturing these lubricants does not produce any side products, because the reaction product of the trans-esterification may be straightaway used as lubricant.

The compound according to this invention includes as components a) at least one thermoplastic polymer. A suitable thermoplastic polymer is for example selected from a group consisting of polyvinylchloride (PVC), polypropylene (PP), polyethylene (PE), polyethylene terephthalate (PET), polylactate (PLA), polycarbonate, polystyrolene, polyurethane, polyether, rubber, preferably polyisoprene, latex, especially NBR latex, polybutadiene, copolymers from at least two of the above-mentioned, especially polyethylene/polypropylene copolymers and mixtures from at least two of these. Preferred are PE, PP, PVC, PET and PLA and most preferred is PVC.

In a design according to this invention the invention-specific thermoplastic compound does not melt below 80° C., preferably at or below 140° C. and especially preferred at or below 270° C. Mostly, the invention-preferred thermoplastic compounds are situated around 350° C. or less as melt.

In invention-specific thermoplastic compounds, for at least 50 weight %, preferably at least 75 weight % and especially preferred at least 90 weight %, each referenced to the overall weight of the thermoplastic compound, thermoplastic polymers with their according melting characteristics are used.

A thermoplastic compound according to this invention contains preferably at least 50 weight %, referenced on the overall weight of the thermoplastic compound, of at least one thermoplastic polymer. The upper limit for the thermoplastic polymer content can be at 99.999 weight %, referenced to the overall weight of the thermoplastic compound, or below, possibly at 85 weight %, 80 weight %, 75 weight %, 70 weight %, 65 weight %, 60 weight % or at 55 weight %. It is moreover also possible that a thermoplastic compound according to this invention contains less thermoplastic polymers, for example 45 weight % or less, 25 weight % or less or 20 weight % or less. Depending on the intended use, the lower limit of thermoplastic polymer content can be at more than 15 weight %, for example at 16, 17, 18, 19 or 20 weight %, referencing the overall weight of the thermoplastic compound respectively.

The compound according to this invention has thermoplastic characteristics. This however does not exclude the possibility that an invention-specific thermoplastic compound may possess a share of non thermoplastic polymer components, for example a share of thermoset material (in literature often termed curable plastics), which however may only amount to a share that does not negatively impact the thermoplastic characteristics of the whole compound in such a way as to make the thermoplastic processing impossible or involve grave disadvantages.

The compound according to this invention also contains as component b) at least one lubricant, whereby the lubricant is a reaction mixture, which is preferably obtained by the reaction of a component containing saturated triglyceride with a primary alcohol. This reaction mixture contains partial esters of glycerine, and besides that it contains an ester of fatty acids and the primary alcohol.

As components containing triglycerides, preferably hardened oils and fats of plant or animal origin are used. For the invention task, the use of fats and oils with a triglyceride share of more than 50 weight %, preferably more than 75 weight % and especially preferred more than 90 weight %, referenced to the overall weight of the oil or fat, respectively, is preferred.

Furthermore it is preferred for the purpose of the invention, to use a component containing triglyceride to manufacture the lubricant, the component should have at least 75 weight %, especially preferably 85 weight % and most preferred 95 weight %, referencing the overall weight of the component containing the triglyceride, oils and fats with an iodine value determined according to DIN 53241-1 of less than 20, especially preferably less than 10 and most preferred less than 5.

This type of fats and oils may occur naturally or be manufactured by hydrating of natural fats and oils with a higher share in unsaturated fatty acids and thereby with a higher iodine value. An example for naturally occurring oil and fats with iodine value under 20 could be coconut oil, which differs according to harvest and origin, which means the fatty acid composition of this natural oil may vary. For the invention, only natural coconut oil with iodine values of less than 20, preferably of less than 10, is usable.

Other suitable natural fats and oils with iodine values of less than 20 are manufactured by hydrating the double-bindings of unsaturated fatty acids in natural fats and oils in a known manner. The suitable natural oils and fats for hydrating are palm oil, palm kernel oil, coconut oil, olive oil, rapeseed oil of old and new culture, sunflower oil of old and new culture, linseed oil, peanut oil, cottonseed oil, coriander oil, meadow farm oil, lard oil, beef tallow and fish oil, chaulmoogra oil, castor oil, but especially hardened beef tallow. The hydrated fats with iodine values below 20 as well as the basic natural fats and oils that have to be hydrated, are available commercially. Preferred is the use of coconut oil, palm kernel oil, hardened tallow, hardened rapeseed oil, hardened palm stearin, hardened castor oil, or a mixture of at least two of these oils.

A primary alcohol, which is reacted with the triglyceride containing component when the lubricant is manufactured, is preferably taken to mean an alcohol consisting of at least one structural unit —CH₂—OH. Preferably, this primary alcohol is a monool, a diol or a polyol with more than two OH groups, for example a polyol with three or four OH groups. Preferred primary alcohols are selected from a group consisting of glycol, trimethylolpropane, pentaerythritol, dipentaerythritol, ethylene glycol, diethylene glycol, triethylene glycol, polyethylene glycol, linear or branched monools with more than seven carbon atoms and a mixture of at least two of these.

The component containing triglyceride when transforming the primary alcohol is preferably reacting by transesterification of the triglycerides. For this reason it is preferred, if the reaction of the component containing saturated triglycerides with the primary alcohol is done with a base as catalyst. Suitable bases are especially hydroxide, oxide or carbonate of an alkaline or alkaline earth metal or alkaline alkoxylate, for example sodium methylate, sodium ethylate or alkaline alkoxylate of the previously listed primary alcohols, whereby the preferred base compounds are selected from a group consisting of NaOH, KOH, LiOH or a mixture of at least two of these.

Furthermore, it is preferred in conjunction with the manufacture of the lubricant, if the component containing saturated triglycerides is reacted with the primary alcohol in a weight relation in the area of 50:50 up to 99:1, especially preferred in an area of 60:40 to 98:2 and most preferred in an area of 70:30 to 95:5.

Further, it is preferred in conjunction with the manufacture of the lubricant, that the reaction of the component containing saturated triglycerides with the primary alcohol is done at a temperature in the area between 100 to 300° C., especially preferred in the area between 150 to 280° C., and most preferred in an area between 200 to 250° C.

It has been shown to be especially advantageous for the purpose of this invention, if the reaction mixture obtained after the transesterification, which, beside the transesterification product (ester from fatty acid and primary alcohol) contains also partial ester of glycerine and non-reacted primary alcohol, non reacted triglyceride as well as possibly the base used as catalyst, is used directly as lubricant component b) without further purification.

The previously described lubricant is contained in the invention-specific thermoplastic compound preferably in an amount in the area between 0.001 to 10 weight %, especially preferred between 0.1 to 5 weight % and most preferred between 0.3 to 3 weight %, referencing the overall weight of the thermoplastic compound.

Furthermore, the compound according to this invention can contain further additives as component c), whereby the additive preferably is selected from a group consisting of fillers, pigments, stabilizers, further lubricants, softeners, anti block agents, anti fogging agents, anti statics flame retardant agents, expanding agents, fats, oils, antioxidants, acid traps, nucleation agents, solvents and a mixture of at least two of these.

Suitable pigments are on principle all organic or inorganic pigments that are compatible with the thermoplastic compound as such and especially with the thermoplastic compound in the intended process. Pigments may colour the thermoplastic compound, however pigments are also capable of colouring the thermoplastic compound white or black. Especially suitable pigments are titanium dioxide, pigments based on a zircon oxide, barium sulphate, zinc oxide (zinc white) and lithopone (zinc sulphite/barium sulphate), soot, soot-titanium dioxide mixtures, iron oxide pigments antimony oxide, chromium oxide, spinel such as cobalt blue and cobalt green, cadmium sulphite, cadmium selenite, ultramarine blue, or organic pigments, such as azopigments, phthalocyanin pigments, chinacridon pigments, perylene pigments, diketo-pyrrolopyrrol pigments or anthrachinon pigments.

Suitable fillers are especially calcium carbonate, dolomite, gypsum, wollastonite, magnesium oxide, magnesium hydroxide, silicate, china clay, tallow, fibre glass, glass spheres, wooden spheres, mica stone, metal oxide or metal hydroxide, soot, graphite, stone meal, barium sulphate, fibre glass, tallow, kaolin or chalkstone or metal sulphates, for example heavy metal sulphates which have not only a filling or pigmenting effect but also a stabilising effect on the thermoplastic compound.

The share in pigments of filling materials or mixtures of one or more pigments and one or more filling materials in the whole thermoplastic compound can be up to 50 weight %, referencing the thermoplastic compound. Invention specific it is planned for the thermoplastic compound to contain only a small amount of pigment or fillers, for example about 0.5 to 10 or 1 to 5 weight %. It is however also possible, that a thermoplastic compound according to this invention contains larger amounts of pigments or fillers or their mixtures, for example about 10 to about 50 weight % or about 20 to about 40 weight %, referencing the overall weight of the thermoplastic compound.

Stabilisers prevent synthetic materials such as PVC from disintegrating at high temperatures or from chemical changes. They improve the resistance to wear and tear due to weather. Compounds based on lead, calcium, zinc, barium and tin are used.

Further lubricants serve to facilitate the processing of polymers by reducing the friction between the polymer chains and reducing the melt's wall adhesion. Further lubricants frequently used are metal carboxylate, such as lead and calcium stearate and—laureate, which may also be effective as co-stabilisers at the same time.

Softeners add pliability and flexibility to the synthetic material. Many softeners are in the phthalate group (DEHP, DINP and DIDP) as well as adipate and citrate.

Anti-fogging agents serve to prevent the formation of clouding, caused for example by water droplets on the surface. Such anti-fogging agents are published in DE 10 2004 038 980 A1 and Plastics Additives Handbook, 5^{th} edition, Hanser Verlag, page 609 to 626 and may be obtained from Emery Oleochemicals GmbH.

Anti-block agents are additives which prevent or reduce the adhesion (‘blocking“) of coated surfaces to each other or to substrates (for example when stacking and packaging). Depending on airing time, degree of drying, coating layer thickness, pressure or temperature at a certain stress, the separating agents must be selected accordingly, and these agents are usually added to the coating substance and have to surface during the drying phase. Materials used for this purpose include for example paraffin, polyethylene wax, wax ester, silicon oils, stearate, modified silica acid and talc.

As solvents, water or organic solvents may be used such as alcohols, e.g. as polyglycol, especially polyethylene glycol or polypropylene glycol or a mixture of those mentioned before.

According to a special design of the invention-specific thermoplastic compound, it contains

a) 50 to 99.999 weight %, especially preferred 60 to 94.9 weight % and most preferred 70 to 89.7 weight % of at least one thermoplastic polymer,

b) 0.001 to 10 weight % especially preferred 0.1 to 5 weight % and most preferred 0.3 to 3 weight % of at least one lubricant and

c) 0 to 49.999 weight %, especially preferred 5 to 39.9 weight % and most preferred 10 to 29.7 weight % of other additives,

referencing the overall weight of the thermoplastic compound, whereby the sum of components a) to c) must be 100 weight %.

A contribution to the task according to this invention as mentioned before is made by a process to manufacture a thermoplastic compound by bringing into contact at least the following compound components

A) 50 to 99.999 weight %, especially preferred 60 to 94.9 weight % and most preferred 70 to 89.7 weight %, referencing the overall weight of the thermoplastic compound, of at least one thermoplastic polymer;

B) 0.001 to 10 weight %, especially preferred 0.1 to 5 weight %, and most preferred 0.3 to 3 weight %, referencing the overall weight of the thermoplastic compound, of at least one lubricant, whereby the lubricant is a reaction mixture, which has been preferably obtained by reacting a component containing saturated triglyceride with a primary alcohol; and

C) 0 to 49.999 weight %, especially preferred 5 to 39.9 weight % and most preferred 10 to 29.7 weight %, referencing the overall weight of the thermoplastic compound, of at least one additive substance,

whereby the sum of components A) to C) amounts to 100 weight % and whereby those compounds are preferred as thermoplastic polymers, as lubricants and as additives, which have been previously listed in conjunction with the invention-specific thermoplastic compound as preferred thermoplastic polymers, lubricants and additives.

The bringing into contact of components A), B) and C) can in the simplest way be done through dry mixing of the three components (dry mixing process), whereby at least one thermoplastic polymer can be used as pellets. It is also possible to heat at least one thermoplastic polymer beyond its melting point, thereby melting it and then introducing components B) and C) into the melt (melt mixing process). Subsequently the mixture can be cooled down and possibly transformed into granules.

A contribution to the solution of the initially mentioned task is made by a thermoplastic compound obtainable through the previously mentioned process, whereby it is preferred that this thermoplastic compound features the same characteristics as the initially described invention-specific thermoplastic compound.

A contribution to the solution of the initially mentioned task is also made by a process to manufacture a mould, including the process steps:

I) To provide a invention-specific thermoplastic compound or a thermoplastic compound obtainable through the invention-specific process;

II) Heating the thermoplastic compound to the glass transfer temperature of the thermoplastic polymer or to a temperature above the glass transfer temperature of the thermoplastic polymer;

III) Manufacturing a mould from the heated thermoplastic compound manufactured in process step II.

In step I) of the process according to the invention to manufacture a mould, first an invention-specific thermoplastic compound or a thermoplastic compound obtainable through the invention-specific process is provided.

Then, in process step II), the thermoplastic compound is heated to the glass transfer temperature of the thermoplastic polymer or to a temperature above the glass transfer temperature of the thermoplastic polymer. Hereby it is preferred to proceed heating the thermoplastic compound to a temperature in an area between 5 degree below the glass transfer temperature (T_{g}) up to 100° C. above the glass transfer temperature of the thermoplastic polymer used, especially preferred heating the thermoplastic compound to a temperature in an area between 1 degree below the glass transfer temperature (T_{g}) up to 50° C. above the glass transfer temperature of the thermoplastic polymer used, and most preferred is heating the thermoplastic compound to a temperature in an area between 1 degree above the glass transfer temperature (T_{g}) up to 20° C. above the glass transfer temperature of the thermoplastic polymer used, however here as well as before the upper limit of the temperature area is determined mainly by the disintegration temperature of the thermoplastic polymer used.

In principle, the process steps I) and II) can be done either simultaneously or consecutively. A simultaneous processing of process steps I) and II) may be useful, if the thermoplastic compound is manufactured by means of a melt mixing process. It may be advantageous to transfer the compound produced by the melt mixing process directly into a mould. A consecutive arrangement of process steps I) and II) may be useful, if the thermoplastic compound is manufactured by way of dry mixing processor if the thermoplastic compound has been manufactured by way of the melt mixing process but is not directly made into a mould but rather cooled down first.

In process step III) of the invention-specific process to manufacture a mould, the heated thermoplastic compound made in process step II) is made into a mould, which preferably is a container, a foil, a fibre, a profile or a tube. Processes to manufacture a mould can be injection moulding, extrusion moulding, compression moulding, layer moulding, lamination moulding, hollow moulding, vacuum moulding and transfer moulding, whereby injection moulding is especially preferred.

Furthermore it conforms to a design of the invention-specific process to manufacture a thermoplastic mould, that in at least one further process step IV) at least one part of the mould obtained in step II) serves as mould sample and is reduced in its mass cross section. The mass cross section is the cross section of an area of the mould, which is consisting solidly of the invention-specific thermoplastic mould mass. For example, for containers or casks the mass cross section is the thickness of a container or cask wall. For moulds that are rather fibre- or skein shaped the mass cross section is the thickness of the threads or skeins.

For flat shapes such as plates, layers, sheets, films or foils, the mass cross section is the strength of these flat shapes. For reducing the mass cross section, in principle all suitable methods known to the expert are useable. For example, stretching in one or two directions, pulling in one or two directions, spinning or blowing, done preferably at elevated temperatures, at which the invention-specific compound is soft or even liquid enough to permit stretching, pulling, spinning or blowing. The part area where the cross section is to be reduced is preferably at least 50% and especially preferred 80% of the mould obtained in step III). In general, stretching and pulling is done, when the mould obtained in step III) is to be made into a fibre. When manufacturing a foil, pulling and stretching may be done in one or more dimension. A sheet coming out of an extruder may be pulled to a roller at a higher speed compared to the output speed from the extruder. If however a container or cask is to be obtained, then besides stretching, pulling and spinning mainly blowing is done in step IV). Hereby the reduction of mass cross section is effected by introducing a gas pressure. The gas pressure is selected in such a way, that the thermoplastic compound of the mould obtained in step III) which has mostly been heated at least to glass transfer temperature, can be stretched. Normally, the expansion is limited by using a form to shape the mould. It is also possible to complement two or more process steps I) to IV) by further process steps and/or creating a time overlap. This is especially so for process steps III) and IV).

A contribution to solve the initially mentioned tasks is also provided by a mould obtained through the previously described process.

A contribution to the solution of the earlier described tasks is made by using the reaction mixture described in conjunction with the invention-specific thermoplastic compound, which is preferably obtainable through reaction of a component containing saturated triglyceride with a primary alcohol, or preferably has already obtained, as a lubricant in thermoplastic compounds. Those which have been identified initially in conjunction with the invention-specific thermoplastic compound as preferred thermoplastic polymers and lubricants are preferred as thermoplastic polymers and as lubricants.

The solution is now explained in closer detail using non-limiting examples.

### EXAMPLE 1

### Transesterification of Hardened Tallow with Trimethylolpropane

In a glass flask, 518.3 g hardened tallow (iodine value <1), 81.7g trimethylolpropane and 0.061 g LiOHxH₂O were introduced and heated to 240° C. while stirring. After 2.5 hrs reaction time, cooling commenced.

Colour 1″ Lov. Yellow =0.5, Lov. red=0.2(lov.=colour code according to Lovibond)

Acid value=0.27, saponification value=171.2

### EXAMPLE 2

### Transesterification of Hardened Tallow with Pentaerythritol

In a glass flask, 517.2 g hardened tallow (iodine value <1), 82.8 g pentaerythritol and 0.06 g LiOHxH₂O were introduced and heated to 240° C. while stirring. After 2 hrs reaction time, cooling commenced. Colour 1″ Lov. Yellow=0.7, Lov. red=0.3

Acid value=0.22, saponification value=170.0

### EXAMPLE 3

### Transesterification of Hardened Tallow with Triethylene Glycol

In a glass flask, 510.0 g hardened tallow (iodine value <1), 90.0 g triethylene glycol and 0.06 g LiOHxH₂O were introduced and heated to 240° C. while stirring. After 2.5 hrs reaction time, cooling commenced.

Colour 1″ Lov. Yellow=3.8, Lov. red=1.4

Acid value=0.8, saponification value=171.3

### EXAMPLES 4-7

### Manufacturing a Thermoplastic Compound According to this Invention

From PVC powder and various additives, a dry blend was produced in a mixer made by Company Henschel. (Material amount=3 kg, heating temperature=120° C., subsequent cooling), the composition is to be taken from the following table.

| Example | 4 | 5 | 6 | 7 |
|---|---|---|---|---|
| PVC Evipol SH 6520 | 100 | 100 | 100 | 100 |
| Lead sulphate 3-base | 3 | 3 | 3 | 3 |
| Lead stearate 28% | 0.5 | 0.5 | 0.5 | 0.5 |
| Calcium stearate | 0.5 | 0.5 | 0.5 | 0.5 |
| Distearyl phthalate | 1 | | | |
| Transesterification product from example 1 | | 1 | | |
| Transesterification product from example 2 | | | 1 | |
| Transesterification product from example 3 | | | | 1 |

The examples 5 to 7 are according to this invention.

### Manufacture of Ribbon Cables as Moulds

The dry-blends from examples 4 to 7 are extruded to a ribbon cable on a double screw extruder made by Company Weber (Parameters of extrusion: rotation speed=15 rpm (B1-B3), 25 rpm; temperature=180° C.). Measurement evaluation for lubricant effect is the extruder's input, the machine load (in %) and the melt pressure (in bar).

| Example | Machine load (in %) | Melt pressure (in bar) |
|---|---|---|
| 4 | 49 | 382 |
| 5 | 46 | 362 |
| 6 | 48 | 365 |
| 7 | 49 | 376 |

The products according to this invention are comparable to the standard lubricant Distearyl phthalate and lower the melt pressure in the extruder without altering the machine load.

The extruded product is used to determine the thermal stability according to EN 60811-3-2:1995 section 9 (Congo red test) at 200° C.

| Example | Stability (min) |
|---|---|
| 4 | 46 |
| 5 | 51 |
| 6 | 51 |
| 7 | 51 |

The lubricants according to this invention achieve a better thermal stability than the standard.

1. A thermoplastic compound comprising
a) at least one thermoplastic polymer, and
b) at least one lubricant,
wherein the at least one lubricant is a reaction mixture obtained by reacting a component which contains saturated triglycerides with a primary alcohol.

2. The thermoplastic compound according to claim 1, wherein the component containing triglyceride is selected from a group consisting of coconut oil, palm kernel oil, hardened tallow, hardened rapeseed oil, hardened palm stearin, hardened castor oil and a mixture of at least two of these.

3. The thermoplastic compound according to claim 1, wherein the component containing triglyceride, for at least 75 weight %, referencing the overall weight of components containing triglycerides, is based on oils and fats with an iodine value lower than 20.

4. The thermoplastic compound according to claim 1, wherein the primary alcohol is a monool, diol or a polyol with more than two OH groups.

5. The thermoplastic compound according to claim 4, wherein the primary alcohol is selected from a group consisting of glycol, trimethylolpropane, pentaerythritol, dipentaerythritol, ethylene glycol, diethylene glycol, triethylene glycol, polyethylene glycol, linear or branched monools with more than 7 carbon atoms and a mixture of at least two thereof.

6. The thermoplastic compound according to claim 4, wherein the primary alcohol is selected from a group consisting of glycol, trimethylolpropane, pentaerythritol, dipentaerythritol and a mixture of at least two thereof

7. The thermoplastic compound according to claim 1, wherein the reaction of the component containing saturated triglycerides with the primary alcohol takes place in the presence of a base.

8. The thermoplastic compound according to claim 7, wherein the base is a hydroxide, an oxide or a carbonate of an alkali or alkaline earth metal or an alkali-alkoxylate.

9. The thermoplastic compound according to claim 8, wherein the base is selected from a group consisting of NaOH, KOH, LiOH and a mixture of at least two thereof

10. The thermoplastic compound according to claim 1, wherein the component containing saturated triglyceride is reacted with the primary alcohol in a weight proportion within an area of 50:50 to 99:1.

11. The thermoplastic compound according to claim 1, wherein the reaction of the compound containing saturated triglycerides with the primary alcohol takes place at a temperature within an area of 100 to 300° C.

12. The thermoplastic compound according to claim 1, wherein the thermoplastic polymer is selected from the group consisting of polyvinylchloride, polypropylene, polyethylene, polyethylene terephthalate, polylactate, polycarbonate, polystyrene, polyurethane, polyether, rubber and copolymers consisting of at least two of the above mentioned.

13. The thermoplastic compound according to claim 1 further comprising an additive, wherein the additive is selected from a group consisting of filler materials, pigments, stabilizers, other lubricants, diluents, anti-block agents, anti-fog agents, antistatic agents, flame retardant agents, propellants, fats, oils, antioxidants, acid neutralizers, nucleating agents, solvents and a mixture of at least two thereof.

14. The thermoplastic compound according to claim 1, wherein the compound contains the lubricant in an amount of about 0.001 to about 10 weight %, referencing the overall weight of the thermoplastic compound.

15. The thermoplastic compound according to claim 1, wherein the thermoplastic compound contains:
a) minimum 50 to 99.999 weight % of the at least thermoplastic polymer,
b) 0.001 to 10 weight % of at least one lubricant and
c) 0 to 49.999 weight % of the other additives,
referencing the overall weight of the thermoplastic compound, wherein the sum of components a) to c) is 100 weight %.

16. A process to manufacture a thermoplastic compound by bringing into contact at least the following compound components:
A) a minimum of 50 to 99.999 weight %, referencing the overall weight of the thermoplastic compound, of at least one thermoplastic polymer;
B) 0.001 to 10 weight % referencing the overall weight of the thermoplastic compound, of at least one lubricant, wherein the lubricant is a reaction mixture, obtainable through reacting one component containing saturated triglyceride with a primary alcohol; and
C) 0 to 49.999 weight %, referencing the overall weight of the thermoplastic compound, of at least one additive
wherein the sum of components A) to C) amounts to 100 weight %.

17. A thermoplastic compound, obtainable through the process according to claim 16.

18. A process to manufacture a moulding, including the following process steps:
I) providing a thermoplastic compound according to claim 1;
II) heating the thermoplastic compound to the glass transition temperature of the thermoplastic polymer or to a temperature above the glass transition temperature of the thermoplastic polymer;
III) manufacturing a moulding from the heated, thermoplastic compound manufactured in process step II).

19. The process according to claim 18, wherein in another process step IV) at least one part area of the moulding obtained in process step III) is reduced in its diameter as opposed to process step III).

20. The process according to claim 18, wherein the moulding is selected from a group consisting of a container, a foil, a fibre, a profile and a tube.

21. A moulding, obtainable through a process according to claim 18.

22. The use of a reaction mixture obtained through a reaction of a component containing saturated triglyceride with a primary alcohol, as a lubricant in thermoplastic compounds.
